(19) United States
(12) Patent Application Publication
(10) Pub. No.: US 2025/0137170 A1
(43) Pub. Date: May 1, 2025

(54) FIBERS WITH EMBEDDED PARTICLES AND METHODS OF PRODUCING FIBERS WITH EMBEDDED PARTICLES

(71) Applicant: Cupron Inc., Richmond, VA (US)

(72) Inventor: Vikram Kanmukhla, Glen Allen, VA (US)

(73) Assignee: Cupron Inc., Richmond, VA (US)

(21) Appl. No.: 18/692,668

(22) PCT Filed: Sep. 16, 2022
(86) PCT No.: PCT/US2022/043828

§ 371 Date: Mar. 15, 2024

(51) Int. Cl. D01F 1/10; A61L 15/18; A61L 15/24; A61L 15/26; A61L 15/46; D01F 6/62; D04H 1/435

(52) U.S. Cl. CPC D01F 1/103; A61L 15/18; A61L 15/24; A61L 15/26; A61L 15/46; D01F 6/62; D04H 1/435; A61L 2300/404; A61L 2300/408; A61L 2300/60; D10B 2401/13; D10B 2509/022; D10B 2509/026

(57) ABSTRACT

Polymeric articles including fibers, films, and solid surfaces may be produced with improved properties by embedding particles within the article. The particles may be any particle that imparts desired properties to the fiber, film, or solid surface. Methods of selecting the particle size based upon the desired properties of the article such as the fiber diameter, film thickness, efficacy such as antimicrobial activity of the article, and/or article compositions are also described. For example, fibers having improved antimicrobial, antiviral, and antifungal properties are described. The articles having antimicrobial, antiviral, and antifungal properties may comprise a plurality of particles comprising water insoluble copper compounds that release at least one of Cu+ ions and Cu++ ions upon contact with a fluid. The average particle size or the particle size range is selected based upon the diameter of the fiber or thickness of the film to be produced.

### FIELD OF THE INVENTION

Articles including fibers, films, and solid surfaces may be produced with improved properties by embedding particles within the article. The particles may be any particle that imparts desired properties to the fiber. Methods of selecting the particle size based upon the desired properties of the article such as the fiber diameter, film thickness, efficacy such as antimicrobial activity of the article, and/or article compositions are also described.

For example, fibers having improved antimicrobial, antiviral, and antifungal properties are described. Embodiments of the articles having antimicrobial, antiviral, and antifungal properties may comprise a plurality of particles comprising water insoluble copper compounds that release at least one of Cu+ ions and Cu++ ions upon contact with a fluid. The average particle size or the particle size range is selected based upon the diameter of the fiber to be produced. In embodiments of the fibers, the particles have an average particle size and/or a particle size range to fiber diameter ratio. The average particle size and/or particle size range or distribution may be selected to maximize the weight of the available portion of the particles. Additional embodiments of the fiber may be produced based on a ratio of the average fiber diameter and the maximum particle size or the minimum particles size.

Embodiments of a method comprises determining a polymeric fiber diameter and polymeric fiber composition for producing a desired product. The product may comprise a fiber, yarn, woven material, fabric, non-woven material. The method may further comprise selecting antimicrobial, antiviral, and antifungal particles or other particles based upon the polymeric fiber diameter. In additional embodiments, the method may comprise selecting an antimicrobial, antiviral, and antifungal particles or other particles based upon the polymeric fiber diameter and the polymeric fiber composition.

### SUMMARY

The properties of polymeric articles including fibers, films, and solid surfaces may be improved or changed by embedding particles having the desired properties within the article. However, proper selection of the particles can optimize the desired properties and reduce the deleterious effects of adding the particles to the polymeric material. The particle composition may be any composition that imparts on or more desired property to the article. The particle size range, maximum particle size, average particle size of the particle, or combinations of these properties may be selected based upon the physical properties of the desired article to be produced. Methods of selecting the particle size based upon the desired properties of the article such as the fiber diameter, film thickness, efficacy such as antimicrobial activity of the article, and/or article compositions are described.

The features of a polymeric fiber that are discernible to the human eye constitute its macrostructure. These macrostructure features include width, length, and crimp. The fiber size is one of the most important properties of fibers. A fiber is typically defined by its diameter or linear density. The size of natural fibers is often given as a diameter in micrometer units. The diameter is an average width along the fiber's length. A fiber size may also be defined by denier, which specify the linear density based on weight of the fiber per unit length.

Embodiments of an antimicrobial, antiviral and antifungal fiber are produced with a desired diameter based upon the processing parameter of the fiber extrusion. Polymeric fibers will comprise an average diameter. The properties of the polymeric fiber may be altered by embedding particles into the polymeric fiber during the production process. Surprisingly, the properties of the embedded may in some applications be improved by providing particles with an average particle size or in a particle size range based upon the average polymeric fiber diameter.

An embodiment of a polymeric fiber, bead, or other article that may be defined by an average diameter may comprise a plurality of first particles having an average particle diameter, wherein the average diameter of the plurality of first particles is in the range of 5% and 10% of the average diameter of the polymeric fiber, bead, or other article.

In another embodiment, the plurality of particles may be chosen based upon the maximum particle diameter of the plurality of particles. In such an embodiment, the polymeric fiber, bead or other article that may be defined by an average diameter may comprise an plurality of particle wherein the maximum particle diameter of the plurality of particles is in the range of 5% and 10% of the average diameter of the polymeric fiber.

In a more specific embodiment, of a polymeric fiber, bead, or other article that may be defined by an average diameter may comprise a plurality of first particles having an average particle diameter, wherein the average diameter of the plurality of first particles and the maximum particle diameter of the plurality of particles are in the range of 2% and 30% of the average diameter of the polymeric fiber, bead, or other article. For smaller average diameter polymeric fibers, the wherein the average diameter of the plurality of first particles and the maximum particle diameter of the plurality of particles are in the range of 3% and 20% of the average diameter of the polymeric fiber, bead, or other article. For fine average diameter polymeric fibers, the wherein the average diameter of the plurality of first particles and the maximum particle diameter of the plurality of particles are in the range of 35% and 15% of the average diameter of the polymeric fiber, bead, or other article.

Further, the plurality of first particles may have a particle size range with a distribution of particle sizes within the particle size range and 90% of the particles have a size which is within the range of 5% and 20% of the average diameter of the fiber.

The polymeric fiber, bead, or other article may comprise any polymeric material. Embodiments of a polymeric fiber may include a polyester fiber or a polypropylene fiber, for example. In one exemplified embodiment, the plurality of particles are particles comprise water insoluble copper compounds that release at least one of Cu+ ions and Cu++ ions upon contact with a fluid.

An embodiment of an incontinence device may comprise a nonwoven top sheet comprising polymeric fibers wherein the polymeric fibers comprise a plurality of particles embedded within the polymeric fibers and the particles comprise water insoluble copper compounds that release at least one of Cu+ ions and Cu++ ions upon contact with a fluid, and an average diameter of the plurality of particles is in the range of 5% and 10% of an average diameter of fibers of the nonwoven top sheet. The polymeric material of the polymer fibers in an incontinence device may be one of a polyester fiber and a polypropylene fiber, for example.

A method of producing antimicrobial, antiviral and antifungal fibers may comprising providing a fiber selection comprising a desired polymeric material and a desired average fiber diameter, selecting a plurality of first particles having an average particle diameter that is between 5% and 20% of the desired average fiber diameter, and extruding a slurry comprising the polymeric material and the plurality of particle into fibers of the desired average fiber diameter.

An embodiment of the method may further comprise determining the concentration of particles in the fiber based upon the average fiber diameter and the average particle diameter of the plurality of particles. Another step in embodiments of the method may comprise determining the maximum loading (concentration) of particles in the fiber based upon the average fiber diameter and the average particle diameter of the plurality of particles.

Another embodiment of the method may comprise a method of producing an antimicrobial, antiviral and antifungal fiber or other articles comprising assessing a desired polymeric fiber, wherein the desired polymeric fiber has a desired fiber composition and a desired average fiber diameter. Then selecting a plurality of first particles having a particle size distribution between 5% and 20% of the desired average fiber diameter and extruding a slurry of the polymeric material and the first particles into fibers of the desired average fiber diameter.

Another method of producing an antimicrobial, antiviral and antifungal fibers or other articles may comprise assessing a desired polymeric fiber, wherein the desired polymeric fiber has a desired fiber composition and a desired average fiber diameter; selecting a plurality of first particles having the desired properties for the fiber and a particle size distribution based upon an algorithm dependent on the desired average fiber diameter; and extruding a slurry of the first particles and the polymeric material into fibers of the desired average fiber diameter.

### BRIEF DESCRIPTION OF THE DRAWINGS

FIG. 1 depicts a cross-sectional view of a polymeric fiber 10 that may be used to produce a yarn, fabric or nonwoven material;

FIG. 2 depicts a cross-sectional view of a polymeric fiber 10 comprising active particles 13A and 13B in an active annular portion and passive particles 13C in a passive core;

FIG. 3 is a graph of various copper oxide loading levels embedded in a ten (10) micron fiber as shown in Table 4 versus the maximum loading of the various sizes of copper oxide particles, the polymeric fibers comprising particles in a particle size range of the copper oxide particles between 0.5 and 2.0 microns had the highest ion availability; the percentage of available copper in the polymeric fiber based upon the copper loading, the percentage of active copper available as a weight percentage of the total copper loading, and total available copper for 10 micron average diameter fiber;

FIG. 4 is a graph of various copper oxide loading levels embedded in a fourteen (14 §) micron fiber as shown in Table 5 versus the maximum loading of the various sizes of copper oxide particles, the polymeric fibers comprising particles in a particle size range of the copper oxide particles between 0.5 and 2.5 microns had the highest ion availability; the percentage of available copper in the polymeric fiber based upon the copper loading, the percentage of active copper available as a weight percentage of the total copper loading, and total available copper for 14 micron average diameter fiber; and

FIG. 5 is a graph of various copper oxide loading levels embedded in a eighteen (18) micron fiber as shown in Table 6 versus the maximum loading of the various sizes of copper oxide particles, the polymeric fibers comprising particles in a particle size range of the copper oxide particles between 0.5 and 3.5 microns had the highest ion availability; the percentage of available copper in the polymeric fiber based upon the copper loading, the percentage of active copper available as a weight percentage of the total copper loading, and total available copper for 18 micron average diameter fiber.

### DESCRIPTION OF THE INVENTION

As way of example, articles having antimicrobial, antiviral, and antifungal properties are described. The articles comprise an antimicrobial, antiviral and antifungal material that kills and/or controls the growth of at least one of microbes, bacteria, molds and fungi. The antimicrobial article may also be used to reduce odor, prevent degradation of to inhibit the catalytic hydrolysis of urea in urine by urease, provide flame retardant properties, or other properties. In embodiments, polymeric articles may comprise a concentration of particles of water insoluble copper compounds that release at least one of Cu+ ions and Cu++ ions upon contact with a fluid (hereinafter “antimicrobial copper compounds”) embedded and/or protruding from the surface of the polymeric fibers exhibit antimicrobial, antiviral and/or antifungal properties in addition to other beneficial properties. Thus, polymeric materials may be converted into products having such properties including, but not limited to, fibers, yarns, films, and nonwoven fabrics through extrusion processes, for example.

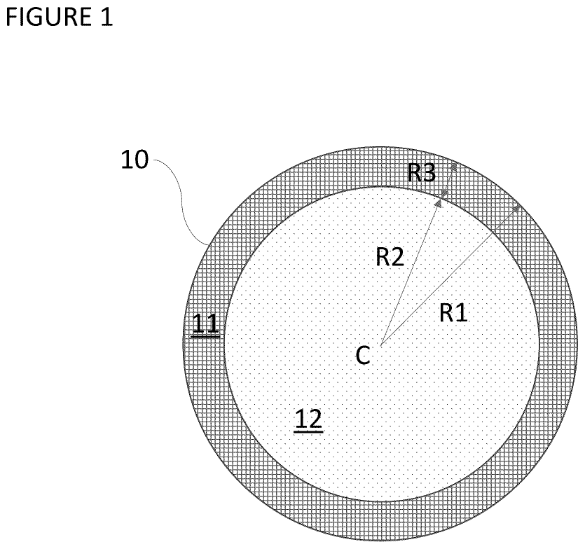

The efficacy and durability of the efficacy of the polymeric articles depends on the amount of the particles that are available to the environment exterior to the material to provide the desired property. For example, FIG. 1 depicts a cross-sectional area of a fiber 10 that may be used to produce a yarn, fabric or nonwoven material. Such polymeric fibers may produced by extruding a molten polymeric material through a spinneret to produce fibers of the desired average diameter. A thin, typically and substantially cylindrical, stream of viscous polymer exits the spinneret into air or liquid to cool the extruded polymer and solidify into the fiber. The method of forming a polymeric fiber may include extrusion, structure setting and drying, and, optionally, a mechanical drawing process.

In a fiber extruded through a spinneret, the individual polymer chains tend to be align. Alignment of molecular chains within polymers is a desirable trait for many applications as it results in superior mechanical and thermal properties in the polymeric materials.

A process for making embodiments of the fibers having embedded particles for adding additional properties to the fiber comprises preparing a polymeric slurry by introducing a plurality of the particles to polymer. The particles may be of the size between 0.1 and 10 microns, for example. The inventor has discovered that different size particles will have significantly different availability to the environment of the fibers. Embodiments of the process further comprise dispersing the particles in the slurry and extruding the polymeric slurry to form polymeric fibers. Preferably, the particles are substantially evenly distributed throughout the polymer slurry and, thus, in the resultant polymer fiber.

The particles are thus incorporated and embedded in the polymer fiber and have a different availability based upon their location within the fiber. A portion of the particles are exposed and protruding from the surface of the material while many other particles are completely embedded within the polymer. Therefore, in some applications, some of the particles that are completely embedded within the polymer may not provide the desired properties to the resulting fiber. For example, the fiber 10 shown in FIG. 1 has a diameter R1. However, there is an active annular portion 11 of particle 10 and a passive core 12. The passive core 12 may comprise some active particles but most particles within the passive core 12 will be embedded and not readily accessible to the outer surface of the fiber to provide properties to the fiber. The particles in the active annular portion 11 will readily provide properties to the fiber 10. Of course, fibers do not have perfectly circular cross-sections and the active annular portion will not be a perfect annular region and the passive core 12 may not have a perfectly circular cross-sectional area. Generally, the active annular portion 11 may be defined as a certain distance R3 from the potentially irregular surface of the fiber. Further, the properties of the polymer including, but not limited to, density, hydrophilicity, hydrophobicity, polymer chain length, composition, may affect the distance R3 and the ratio of the distances of the active annular portion R3 and the passive core R3. FIG. 1 merely depicts a cross-section of an idealized fiber 10. The depth of the annular portion may depend on the depth that moisture may reach within the fiber.

In an embodiment of a polymeric fiber, bead, or other article that may be defined by an average diameter, the polymeric fiber, bead, or other article may comprise a plurality of particles having an average particle diameter, wherein the average diameter of the plurality of first particles and/or the maximum particle diameter of the plurality of particles are in the range of 2% and 25% of the average diameter of the polymeric fiber, bead, or other article. For smaller average diameter polymeric fibers, the wherein the average diameter of the plurality of first particles and the maximum particle diameter of the plurality of particles are in the range of 3% and 20% of the average diameter of the polymeric fiber, bead, or other article. For fine average diameter polymeric fibers, the wherein the average diameter of the plurality of first particles and the maximum particle diameter of the plurality of particles are in the range of 35% and 15% of the average diameter of the polymeric fiber, bead, or other article.

Further, the plurality of particles may have a particle size range with a distribution of particle sizes within the particle size range and 90% of the particles have a size which is within the range of 5% and 20% of the average diameter of the fiber.

In one example, a one (1) denier per filament (hereinafter “dpf”) polyethylene terephthalate polymer fiber may have an approximately one (1) micrometer R3 annular active portion 11 and an approximately four (4) R2 micrometer passive core 12. In such an embodiment, the active estimated annular portion 11 occupies thirty-six percent (36%) of the entire cross-sectional area of the fiber. The passive core 12, therefore, is estimated to occupy 64% of the cross-sectional area of the fiber. Table 1 provides one embodiment of a polymeric fiber showing the percentage of the fiber cross-section that is active for polymeric fibers of 1 dpf, 2 dpf, and 3 dpf with an approximately one (1) micron active area depth from the surface of the fiber.

TABLE 1
Estimated Percentage of Active Ar§ea of various diameter fibers
PET Yarn Properties* (Constant Denier)

| | Fiber | Inner circle | |
|---|---|---|---|
| | Diameter | Diameter | % Active |
| DPF | (micron) | (micron) | Volume |
| 1.00 | 10.00 | 8.00 | 36% |
| 2.00 | 14.14 | 12.14 | 26% |
| 3,00 | 17.32 | 15.32 | 22% |

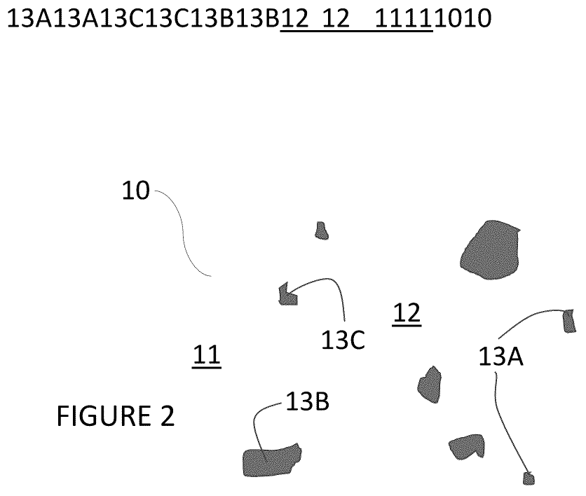

An embodiment of a similar polymeric fiber 10 comprising active particles 13A and 13B and passive particles 13C is depicted in FIG. 2. As in FIG. 1, the polymeric fiber has an active annular portion 11 and a passive core 12. The particles 13A 13B and 13C are embedded in the polymeric fiber. As shown, a portion of the particles are substantially active particles 13A completely embedded in the active annular ring 11. These substantially active particles 13A will provide their properties to the environment outside the polymeric fiber. These substantially active particles 13A are almost entirely available. Particles 13A may be substantially extracted from the polymeric fiber even if they are completely embedded in the annular active portion. The at least partially available particles 13B bridge between the active annular portion 11 and the passive core 12. As the particle is used from the active annular portion 11 at least a portion of the particle 13B extending into the passive core 12 is available to the environment outside of the fiber. Finally, passive particles 13C are not significantly available to the environment outside the fiber and, therefore, do not significantly provide the desired properties. If the fiber is damaged, torn, or worn particles in the passive core may become available to provide the desired properties to the fiber.

Polymeric fibers with embedded antimicrobial copper compounds have antimicrobial, antiviral and antifungal properties. An experiment was designed and performed to determine the percentage of copper oxide particles embedded in the fiber that are bioavailable. Bicinchoninic acid (Cu-BCA) is an excellent chelating agent for cuprous ions and was used to determine the available copper in the fiber. BCA can be used to readily extract the bioavailable copper ions from the polymeric fiber while leaving the passive particles in the fiber. A solution of Cut-BCA has a purple color that can readily be analyzed to quantify the copper in solution using UV-Vis spectrophotometer (at 562 nm) to determine the amount of extracted and thus available copper ions.

### Example 1: Determining Bioavailable Copper Ions in Polymeric Fibers

14.972 grams of a sample polyester fabric comprising 2.58 wt. % of antimicrobial, antiviral, and antifungal particles of Cu₂O (2.29 wt. % of copper) was prepared. The total weight of copper in the sample fabric was 342.8 milligrams. The fabric was soaked in BCA four times to extract all of the bio-available copper ions from the fabric. Analysis of the fluid determined that 24.3 milligrams of copper was extracted. Demonstrating that 7.01% of the copper embedded in the fabric may be extracted and, therefore, bio-available. The results of each exposure to BCA is shown in Table 2.

TABLE 2
Copper ions extracted from fabric in each of four exposures to BCA

| | Cu extracted for | Cumulative Cu |
|---|---|---|
| Exposure | each exposure | extracted |
| No. | mg | mg |
| 1 | 14.0 | 14.0 |
| 2 | 5.1 | 19.1 |
| 3 | 3.5 | 22.6 |
| 4 | 1.7 | 24.3 |

The efficacy of the fabric to kill bacteria was tested to confirm that the bio-available copper had been extracted completely. The results of the efficacy tests are shown in Table 3. The untreated fabric embedded with copper oxides significantly reduced Candida albicans after only a two hour exposure while the fabrics that had been treated to remove the bio-available copper ions showed no reduction in bacterial colonies after the two hour period.

TABLE 3
Efficacy of treated and untreated fabrics on Candida albicans after
two-hour contact time (NR indicates ″NO REDUCTION″).

| SI No. | Sample | Efficacy |
|---|---|---|
| 1 | Untreated Sample Cupron Fabric | 99.81 |
| 2 | Copper Extracted Fabric 1 | NR |
| 3 | Copper Extracted Fabric 2 | NR |

The inventors had shown for the first time that only a specific percentage of the total embedded copper ions are bio-available as antimicrobial particles. Further experiments were run with 1 dpf, 2 dpf and 3 dpf fibers and with copper oxide particles having a particle size range of 0.3 to 0.6 microns (average diameter of 0.45 microns), 1 to 2 microns (average diameter of 1.5 microns), and particles having an average particle size of 0.6 microns. The results demonstrated that fibers having lower dpf with similar particle sizes and similar concentrations of copper oxides have higher weight of bio-available copper available. This result corresponds directly to the hypothesis that each fiber has an active annular portion on the outer surface of the fiber.

Additionally, the results of the experiments demonstrated that fibers comprising larger sized particles (particles with a greater average particle size or greater maximum particle size) and similar fiber diameters and similar concentrations of copper oxides have higher weights of bio-available copper available.

Finally, polymeric fibers loaded with higher concentrations of copper oxide particles had have higher weight of bio-available copper. The higher concentration of copper oxide in the active annular portion and bridging the interface provides additional bio-availability to the copper.

Therefore, the inventor surprisingly discovered that to maximize bio-available copper in a polymeric fiber, the fiber should be loaded with the greatest concentration of the largest particles in the narrowest fibers. This discovery goes against general perception and understanding of one skilled in the art. Prior to this discovery, it was believed in the art that embedded particles in fibers should be nanoparticles because it was believed that you should maximize the surface area of the particles per weight of particles in fiber.

Since, the fiber diameter is typically determined by the application of the fiber. Medical textiles, non-woven sheets including wound dressings and top sheets for incontinence devices, geotextiles, and barrier fabrics may all use different diameter fibers and/or yarns.

The results and the conclusions of the studies may be visualized in FIG. 2. Larger particles will have a higher probability of being available particles, embedded in the active annular portion or protruding from the fiber 13A or bridging the interface between the active annular region and the passive core 13B due to the greater ratio of the average particle diameter to the fiber diameter.

However, there are limits to the practical size of the particles. Larger particles will occupy a significant amount of the cross-sectional area of the fiber and cannot be loaded into a fiber in as high a concentration as a smaller particle. Larger particles weaken the fibers if the particles are loaded above a certain concentration of the weight of the fiber. Generally, larger particles may not be loaded to higher concentrations but a greater percentage of the particles are available at the surface of the fiber.

Conversely, smaller diameter particles may be loaded at greater concentrations than the larger particles but a lower percentage of the particles are available. Tables 4 through 6 provide maximum loading levels of various size particles in three different fiber diameters, 10 micron fibers, 14 micron fibers and 18 micron fibers. Such fibers are limited in their carrying capacity for a specific size particle because above the maximum loading the fibers will be too weak or unable to be processed.

TABLE 4
Maximum loading, percent of active particle and total available
copper for 10 micron fiber

| | | | % | Total | Particle |
|---|---|---|---|---|---|
| | Size of the | Max loading | Available | available | size to |
| | particle | possible | Active, | Copper, | fiber size, |
| | (micron) | (wt %) | as wt % | as wt % | % |
| 10 μ | 0.1 | 8.0% | 0.90% | 0.07% | 1% |
| fiber | 0.5 | 4.5% | 4.50% | 0.20% | 5% |
| dia | 1.0 | 3.0% | 8.80% | 0.26% | 10% |
| (1.0 | 1.5 | 1.5% | 13.00% | 0.20% | 15% |
| dpf) | 2.0 | 1.0% | 17.20% | 0.17% | 20% |
| | 3.0 | 0.5% | 25.20% | 0.13% | 30% |
| | 5.0 | 0.1% | 40.00% | 0.04% | 50% |
| | 8.0 | 0.0% | 60.00% | 0.00% | 80% |

TABLE 5
Maximum loading, percent of active particle and total available
copper for 14 micron fiber

| | | | % | Total | Particle |
|---|---|---|---|---|---|
| | Size of the | Max loading | Available | available | size to |
| | particle | possible | Active, | Copper, | fiber size, |
| | (micron) | (wt %) | as wt % | as wt % | % |
| 14 μ | 0.1 | 10.0% | 0.60% | 0.06% | .7% |
| fiber | 0.5 | 5.0% | 3.00% | 0.15% | 3.6% |
| dia | 1.0 | 4.0% | 5.98% | 0.24% | 7.1% |
| (2.0 | 1.5 | 3.0% | 8.90% | 0.27% | 10.7% |
| dpf) | 2.0 | 2.0% | 11.78% | 0.24% | 14.3% |
| | 3.0 | 1.0% | 17.40% | 0.17% | 21.4% |
| | 5.0 | 0.5% | 28.00% | 0.14% | 35.6% |
| | 8.0 | 0.1% | 42.50% | 0.04% | 57.1% |

TABLE 6
Maximum loading,percent of active particle and total available
copper for 18 micron fiber

| | | | % | Total | Particle |
|---|---|---|---|---|---|
| | Size of the | Max loading | Available | available | size to |
| | particle | possible | Active, | Copper, | fiber size, |
| | (micron) | (wt %) | as wt % | as wt % | % |
| 18 μ | 0.1 | 12.0% | 0.52% | 0.06% | 0.5% |
| fiber | 0.5 | 7.0% | 2.58% | 0.18% | 2.8% |
| dia | 1.0 | 5.0% | 5.13% | 0.26% | 5.6% |
| (3.0 | 1.5 | 4.0% | 7.64% | 0.31% | 8.3% |
| dpf) | 2.0 | 3.5% | 10.12% | 0.35% | 11.1% |
| | 3.0 | 2.0% | 15.00% | 0.30% | 16.7% |
| | 5.0 | 0.8% | 24.30% | 0.18% | 27.8% |
| | 8.0 | 0.2% | 37.00% | 0.07% | 44.4% |

In the embodiments shown in the Tables 4 through 6 the fibers release Cu+ ions when exposed to water or water vapor to provide the antimicrobial and antiviral activity of the polymeric material up to the total available amount of copper. The maximum loading possible is defined as the maximum weight of particles, in this case copper oxide, as a percentage of the total fiber weight (100%*wt. loaded Cu₂O/wt. fiber) that can be loaded into a fiber and still produce a functional fiber. The % Available Active is the percentage of the loaded Cu₂O or other particle that is bio-available or active as a weight percentage (100%* weight of active particles/total weight loaded particle.). The Total Available Copper is the weight of the copper that is bio-available or active as a weight percentage of the fiber (100%* weight of active particles/total weight fiber) and is the product of the values of the maximum loading of the copper in the fiber and the percent of the copper that is bio-available. The inventors provided a method of producing strong fibers with a maximum of bio-available particles, for example, antimicrobial, antiviral and antifungal particles.

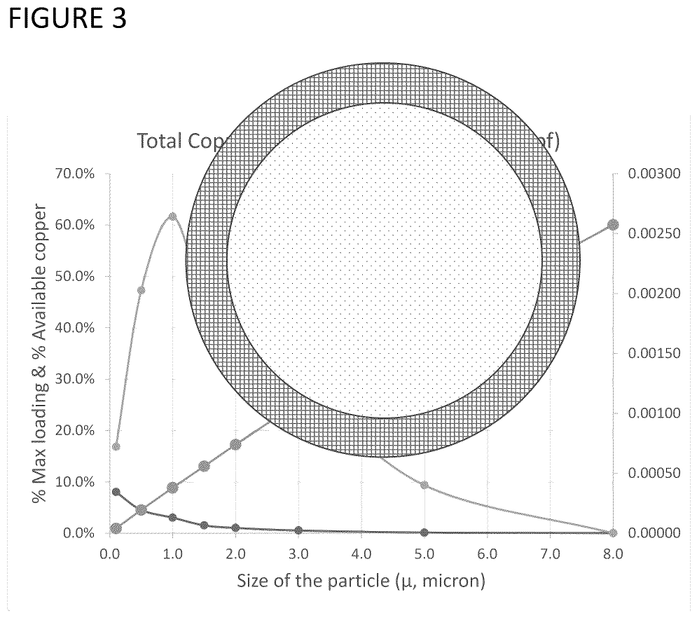

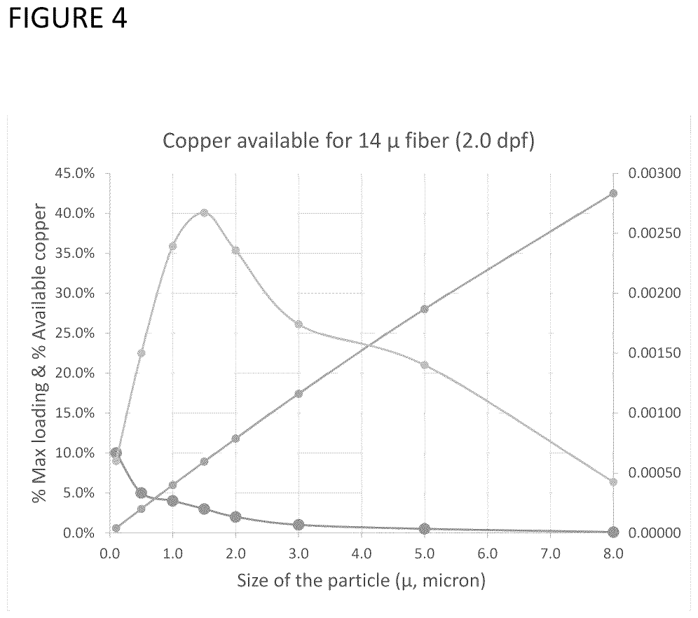

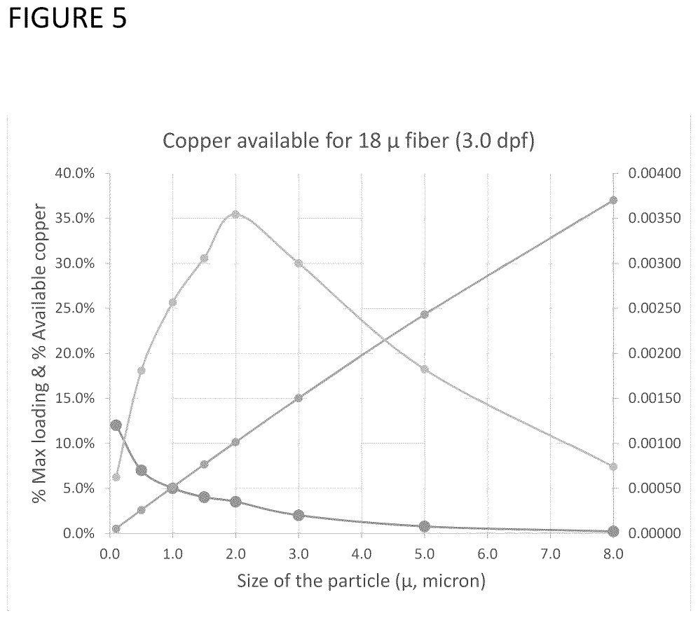

The particle size of the masterbatch or the powder may be chosen to provide the fiber with the maximum available properties or, in the example herein, the maximum available copper to provide antimicrobial, antiviral and antifungal properties to the fiber. FIGS. 3 through 5 are graphs of the data provide in Tables 3 through 5. The maximum bio-available copper for each particle size may be seen for each fiber diameter. In an embodiment of the method of the invention, the method comprises selecting a particle or a masterbatch comprising a having a particle size range (95% of all particles) within a range that extends 1 micron on either side of the particle size with the maximum available weight of particles. For example, in this embodiment, for copper oxide in a fourteen (14) micron fiber as shown in Table 4 and FIG. 4, the particle size range of the copper oxide particles should be between 0.5 and 2.5 microns.

Another embodiment of the method comprises selecting a particle or a masterbatch comprising a particle having a particle size range (at least 95% of all particles) within a range that extends 0.5 micron on either side of the particle size with the maximum available weight of particles. For example, for copper oxide in a ten (10) micron fiber as shown in Table 3 and FIG. 3, the particle size range of the copper oxide particles should be between 0.5 and 1.5 microns. Embodiments of the method of producing the antimicrobial, antiviral, and antifungal articles may comprise adding the particles directly to the polymer as a powder or adding the particles as a component of an antimicrobial copper particle masterbatch. United States Patent Application Publication No. US 2008/0193496 describes a method of producing an antimicrobial and antiviral masterbatch and processes for producing antimicrobial and antiviral masterbatch, and its disclosure is hereby incorporated by reference in its entirety.

Generally, the particle size with the maximum available active material increases for increasing fiber diameter. Thus, larger particles may be used in larger fibers and have greater particle size ranges. In certain embodiments, the particle size range of the plurality of particles may be selected to be within a range from 5% to 20% of the diameter of the fiber. In a more specific embodiment, the, the particle size range of the plurality of particles may be selected to be within a range from 5% to 10% of the diameter of the fiber. In another embodiment, the particle size range may be chosen based upon an algorithm including the fiber diameter. For example, for copper oxide in a polyester fiber, the lower limit of the particle size range in microns may be calculated from the formula [0.5+0.5*(dpf-1)] and the upper limit of the particle size range may be calculated from the formula [1.5+0.5*(dpf-1)]. Other formula may be developed based upon experimental data of different combinations of particles and fiber composition.

A still further embodiment comprises selecting a plurality of particles or a masterbatch comprising a plurality of particles having an average particle size within a range that extends 20% above and below the particle size with the maximum available weight of particles. For example, for copper oxide in a ten (10) micron fiber as shown in Table 3 and FIG. 3, the average particle size of the copper oxide particles should be between 0.8 and 1.2 microns in this embodiment.

In certain embodiments, the antimicrobial copper particles may be added to a melted polymer to form polymeric slurry. The particles may be added to the polymer in a powder for or a masterbatch form. The polymers of the fiber and/or the masterbatch may include, but are not limited to, polyolefins, polyethylene, high density polyethylene, low density polyethylene, polystyrene, polyacrylates, polymethacrylates, polyethylene terephthalate (PET), polybutylene terephthalate (PBT), polytrimethylene terephthalate, polylactic acid (PLA), polyglycolide (PGA), polylactic-co-glycolic acid (PLGA), polyvinylchloride (PVC), polyurethanes, polyacrylates, phenol-formaldehyde (PF), polyamides or nylon including, but not limited to, nylon-6 (polycaprolactum) and Nylon 66, polyurethanes, similar thermoplastic polymers or copolymers, super absorbent polymers, and combinations thereof. Polyesters are polymers formed from a dicarboxylic acid and a diol. The polymer may be extruded to produce fibers, yarns, nonwovens, or sheets which possess antimicrobial, antifungal and/or antiviral properties.

The invention has been primarily exemplified with copper particles, however, the particles may be any particles which provide desired properties to the fiber including, but not limited to, cuprous oxide (Cu₂O), cupric oxide (CuO), cuprous iodide (CuI), cuprous thiocyanate (CuSCN), copper sulfide, copper sulfate (CuSO₄), copper chloride (CuCl₂), zeolites, especially copper-zeolites, zirconium phosphate, copper-zirconium phosphate, zinc pyrithione, zinc oxide, titanium oxide, titanium dioxide, titanium oxide, silver nitrate, silver oxide, and silver oxide, silver iodide, silver chloride, silver sulfate, silver sulfide, quarternary ammonium compounds, and combinations thereof. The particles may be added to a polymer in the process with a polymeric masterbatch comprising the particles.

Based upon the fiber diameter and/or the fiber composition, each masterbatch will comprise a polymer or a blend of polymers and a plurality of antimicrobial particles or other desired particles in a particular particle size range and/or an average particle size. Therefore, the appropriate masterbatch may be chosen based upon the intended fiber composition (polymer, for example) and the desired fiber diameter. In some embodiments, two or more masterbatches may be used to produce a fiber comprising the desired strength, processability, and other properties. The masterbatches may further comprise antimicrobial polymers such as, but not limited to, polyhexamethylene biguanide.

Embodiments of the method of producing the antimicrobial, antiviral, and antifungal fibers may comprise adding the antimicrobial copper particles to the polymer as a powder and/or adding the antimicrobial copper particles as a component of an antimicrobial copper particle masterbatch. Embodiments of a method of producing an antimicrobial, antiviral, and antifungal article include selecting two or more masterbatches from a plurality of masterbatches, wherein the masterbatches comprise antimicrobial copper particles having different particle size ranges, different copper compositions, and/or blends of copper compositions. In such a method, the fibers may be tailored to have a combination of desired properties based upon the chosen antimicrobial copper particles.

Embodiments of the antimicrobial, antiviral and antifungal fibers may comprise a polymeric material, wherein the polymeric material has been extruded into a fiber. The extruded fiber has an average diameter. The fibers also comprise a plurality of first particles having an average particle diameter, wherein the first particles comprise water insoluble copper compounds that release at least one of Cu+ ions and Cu++ ions upon contact with a fluid embedded in the fiber and wherein the average diameter of the particle is in the range of 5% and 20% of the average diameter of the polymeric fiber.

The inventor has determined that by producing fibers with the final fiber diameter determining the selection of the size of the antimicrobial copper particles, the strength and processability of the fibers can be optimized for an application. The selection of a powder or masterbatch may be based upon the desired properties of the fibers including, but are not limited to, strength, extrusion efficiency, antimicrobial, antiviral and antifungal properties, copper ion release rate, particle size, copper structural state including amorphous or crystalline, contact killing properties, optical qualities, thereby tailoring the fibers.

The strength of the fibers may be determined by the polymer, the physical properties of the polymer, the composition of the antimicrobial copper particle, the size of the antimicrobial copper particle, and other physical and chemical properties and interactions. For example, embodiments of the fibers and methods may also comprise selecting the maximum size of the antimicrobial copper particles based upon the desired fiber diameter and the desired fiber strength. In some embodiments, the maximum particle diameter of the plurality of particles may be in the range of 25% and 30% of the average diameter of the polymeric fiber. In other embodiments, ninety or ninety-five percent of the particles of the plurality of particles may be below 25% of the average diameter of the polymeric fiber. In a further embodiment, the plurality of first particles are within a particle size range, and ninety or ninety-five percent of the particles are in the range of 5% and 20% of the average diameter of the fiber. The average particle diameter, the particle size ranges and the fiber diameter are measured by known methods.

Another embodiment includes a method for producing antimicrobial, antiviral and antifungal fibers. Embodiments of the method of producing an antimicrobial, antiviral and antifungal fiber comprise determining a fiber to produce including a desired fiber composition and a desired average fiber diameter. Different size fiber may be desired for different applications due to the tactile feel of the fibers, the strength of the fibers, etc. The method comprises selecting a plurality of particles having an average particle diameter that is between 5% and 20% of the desired average fiber diameter. The particles may be antimicrobial copper particle comprising water insoluble copper compounds that release at least one of Cu+ ions and Cu++ ions upon contact with a fluid. The polymer and the plurality of the particles may be mixed, melted to produce a polymeric slurry, and extruded to the desired average fiber diameter. The desired particles may be mixed with the desired polymer by adding an antimicrobial copper particle masterbatch.

Embodiments of the method may base the selection of the antimicrobial copper particles or the antimicrobial copper particle masterbatch on the maximum diameter of a particle of the plurality of first particles. In embodiments, the maximum particle diameter may be within the range of 5% and 10% of the average diameter of the polymeric fiber.

### Example 2

An incontinence device typically comprises a top sheet, an absorbent core, and a water impermeable bottom sheet. Water insoluble copper compounds that release at least one of Cu+ ions and Cu++ ions upon contact with a fluid may be added to at least one of the top sheet, the absorbent core and the bottom sheet or another component of the diaper to inhibit the catalytic hydrolysis of urea in urine by urease. The water insoluble copper compounds thereby reduce the rate of degradation of the urea to ammonia and carbon dioxide. The antimicrobial copper particles may also reduce the risks of infection of the wearer. By significantly reducing the formation of ammonia, the pH of the environment within the diaper can be maintained slightly acidic and more beneficial to skin health.

In one embodiment, the incontinence device comprises a nonwoven top sheet, wherein the top sheet comprises polymeric fibers. The polymeric fibers comprise a plurality of particles, wherein the particles comprise water insoluble copper compounds that release at least one of Cu+ ions and Cu++ ions upon contact with a fluid embedded in the polymeric fibers and an average diameter of the particle is in the range of 5% and 20% of the average diameter of fibers of the nonwoven top sheet. The polymeric fibers may, typically, be polyester fibers or polypropylene fibers, however, other polymeric materials may also be used. It is known in the art that the nonwoven top sheet may comprises aperture. The composition, physical, and chemical properties of the antimicrobial copper particles adjacent to the apertures may be different than the composition, physical, and chemical properties of the antimicrobial copper particles in the nonwoven top sheet generally. The antimicrobial copper particles may be chosen for their ion release properties or their contact killing properties to tailor the different portions of the top sheet.

### Example 3

Surgical sutures may be produced having the specific properties described herein. A twenty (20) micron PLGA fiber suture may be loaded with copper oxide particle having an average particle diameter of the approximately 2.0 microns, 3.5 wt. % of copper oxide in the fiber, and has 0.35 wt. % of bio-available copper per fiber weight.

### Example 4

A wound dressing typically comprises an absorbent core sandwiched between two top sheets. Water insoluble copper compounds that release at least one of Cu+ ions and Cu++ ions upon contact with a fluid may be added to at least one of the top sheet and the absorbent core to inhibit catalytic hydrolysis of proteins in blood and serum by proteases and lipases. The water insoluble copper compounds thereby reduce the rate of degradation of the blood and serum and other fluid components to form an alkaline environment (ammonia). The antimicrobial copper particles may also reduce the risks of infection of the wearer.

In one embodiment, the wound dressing comprises a nonwoven top sheet, wherein the top sheet and adsorbent core comprises polymeric fibers. The polymeric fibers comprise a plurality of particles, wherein the particles comprise water insoluble copper compounds that release at least one of Cu+ ions and Cu++ ions upon contact with a fluid embedded in the polymeric fibers and an average diameter of the particle is in the range of 5% and 20% of the average diameter of fibers of the nonwoven top sheet. The polymeric fibers may, typically, be polyester fibers or polypropylene fibers, however, other polymeric materials may also be used.

1. An antimicrobial, antiviral, or antifungal polymeric fiber, comprising:
a polymeric material, wherein the polymeric fiber has an average diameter; and
a plurality of first particles having an average particle diameter, wherein the average particle diameter of the plurality of first particles is in the range of 5% and 15% of the average diameter of the polymeric fiber.

2. The antimicrobial, antiviral, or antifungal fiber of claim 1, wherein the polymeric fiber is one of a polyester fiber and a polypropylene fiber.

3. The antimicrobial, antiviral, or antifungal fiber of claim 2, wherein plurality of first particles has a maximum particle diameter and the maximum particle diameter of the plurality of first particles is in the range of 5% and 10% of the average diameter of the polymeric fiber.

4. The antimicrobial, antiviral, or antifungal fiber of claim 1, wherein the plurality of first particles are within a particle size range and 90% of the particles are in the range of 5% and 20% of the average diameter of the fiber.

5. The antimicrobial, antiviral, or antifungal fiber of claim 1, wherein the particles comprise water insoluble copper compounds that release at least one of Cu+ ions and Cu++ ions upon contact with a fluid.

6. An incontinence device, comprising:
a nonwoven top sheet comprising polymeric fibers and the polymeric fibers comprise a plurality of particles embedded within the polymeric fibers and the particles comprise water insoluble copper compounds that release at least one of Cu+ ions and Cu++ ions upon contact with a fluid, and an average diameter of the plurality of particles is in the range of 5% and 10% of an average diameter of fibers of the nonwoven top sheet.

7. The incontinence device of claim 6, wherein the polymeric material is one of a polyester fiber and a polypropylene fiber.

8. The incontinence device of claim 6, wherein the nonwoven top sheet comprises apertures.

9. A method of producing an antimicrobial, antiviral and antifungal fiber, comprising:
providing a fiber selection comprising a desired fiber composition and a desired average fiber diameter;
selecting a plurality of first particles having an average particle diameter that is between 5% and 20% of the desired average fiber diameter; and
extruding the polymeric material into fibers of the desired average fiber diameter.

10. The method of claim 9, comprising determining the concentration of first particles in the fiber based upon the average fiber diameter and the average particle diameter.

11. The method of claim 10, comprising determining the maximum loading of first particles in the fiber based upon the average fiber diameter and the average particle diameter.

12. The method of claim 9, wherein the step of selecting a plurality of first particle comprises selection a masterbatch comprising the plurality of first particles.

13. The method of claim 9, wherein the plurality of first particles comprise water insoluble copper compounds that release at least one of Cu+ ions and Cu++ ions upon contact with a fluid.

14. The method of claim 12, wherein the masterbatch comprises the plurality of first particle and a polymer.

15. The method of claim 9, wherein the maximum diameter of a particle of the plurality of first particles is in the range of 5% and 10% of the average diameter of the polymeric fiber.

16. A method of producing an antimicrobial, antiviral and antifungal fiber, comprising:
providing a fiber selection comprising a desired fiber composition and a desired average fiber diameter;
selecting a plurality of first particles having a particle size distribution between 5% and 20% of the desired average fiber diameter; and
extruding the polymeric material into fibers of the desired average fiber diameter.

17. A method of producing an antimicrobial, antiviral and antifungal fiber, comprising:
providing a fiber selection comprising a desired fiber composition and a desired average fiber diameter;
selecting a plurality of first particles having a particle size distribution based upon an algorithm dependent on the desired average fiber diameter; and
extruding the polymeric material into fibers of the desired average fiber diameter.
